(19) United States
(12) Patent Application Publication
(10) Pub. No.: US 2016/0136197 A1
(43) Pub. Date: May 19, 2016

(54) Pharmaceutical Compositions Comprising a GPG Oligodeoxynucleotide and Cyclic Di-GMP

(71) Applicants: INTERVET INTERNATIONAL B.V., AN Boxmeer (NL); INTERVET INC., Summit, NJ (US)

(72) Inventors: Carla Christina Schrier, Boxmeer (NE); Martijn BROK DEN, Nijmegen (NE); Gosse Adema, Nijmegen (NE)

(21) Appl. No.: 14/898,000

(22) PCT Filed: Jun. 13, 2014
(86) PCT No.: PCT/EP2014/062308

§ 371 Date: Dec. 11, 2015

(30) Foreign Application Priority Data
Jun. 14, 2013 (EP) 13172144.1

(51) Int. Cl. A61K 31/7088; A61K 31/7084

(52) U.S. Cl. CPC A61K 31/7088; A61K 31/7084

(57) ABSTRACT

The present invention relates to pharmaceutical compositions comprising an immunostimulatory amount of at least two immunopotentiators, wherein a first immunopotentiator is a non-methylated cytidyl guanosyl oligodeoxynucleotide (CpG ODN) and a second immunopotentiator is 3′,5′-cyclic diguanylic acid (c-di-GMP), and a pharmaceutically acceptable carrier. The invention also relates to the use of such pharmaceutical compositions for the induction of an immune response against tumor-specific antigens. Also the invention relates to their use in in situ tumor-destruction therapy and to such pharmaceutical compositions for use in the treatment of a mammal suffering from cancer.

The present invention relates to pharmaceutical compositions comprising an immunostimulatory amount of at least two immunopotentiators, to their use for the induction of an immune response against tumor-specific antigens, their use in in situ tumor-destruction therapy and to such pharmaceutical compositions for use in the treatment of a mammal suffering from cancer.

Cancer is a general term, used to describe neoplastic growth. Neoplasms are considered abnormal, usually de-differentiated forms of tissue that commonly proliferate at a higher speed than normal. In most cases neoplastic cells invade surrounding tissue and moreover they metastasize and continue to grow elsewhere in the body.

Local and regional treatment of the neoplastic mass, the tumor, such as surgery, does not affect possible metastases. Therefore, additional therapies are needed such as treatment with cytotoxic drugs. Such treatment is generally known as chemotherapy.

Another approach lies in the induction of a humoral immune response against tumor-specific antigens, often referred to as cancer immunotherapy. The advantage of successful induction of such an immunological response to tumor-specific antigens is that it will last for some time and eventually eliminate tumor localization elsewhere in the body that is not amenable for local tumor treatment. A systematic review of humoral immune responses against tumor antigens is published by Reuschenbach, M. et al., in Cancer Immunol Immunother. 58: 1535-1544 (2009). Sometimes, spontaneous humoral immune responses against one or several self-tumor antigens are reported. In other cases, a humoral immune response is deliberately induced, e.g. through tumor cell isolation, growth of these cells in vitro and subsequent killing of the cells followed by injection into the patient in the presence of an immunostimulator.

Local treatment is of course a first step in the treatment of solid tumors. This is traditionally done by means of tumor resection. Another approach is tumor destruction in situ. A characteristic of tumor destruction in situ is that the tumor is not removed but necrotized. In principle irradiation is a form of tumor destruction in situ, but many other ways of tumor destruction have been developed. Common methods are e.g. photodynamic therapy using the combination of photosensitizing compounds and their subsequent activation by laser, in situ heating by means of laser light, microwaves, electric current, ultrasound, high intensity focused ultrasound or by means of radiofrequency waves, or cryotherapy: necrotizing tissue by freezing.

Tumor destruction in situ leaves the destructed tumor mass present in the body. This leaves the possibility open to try and build an immunological response to tumor-specific antigens (cancer immunotherapy) in the destructed tumor in situ. The advantage of successful induction of such an immunological response to tumor-specific antigens is that no material from the tumor has to be isolated, grown in vitro and re-injected into the patient.

Contrary to what is known from vaccine development that is based upon non-self antigens, the induction of an immunological response against to tumor-specific antigens is far from easy, regardless the method used to induce the immunological response. Basically, tumor antigens are predominantly normal components of the body: self-antigens. Therefore the immune system as such will down-regulate self-directed immune response leading to a tolerant state for self-antigens.

Thus, the development of cancer immunotherapy requires a very specific approach, based on immunopotentiation Immunopotentiation is a general term for the enhancement of the immune response by increasing the speed and/or extent of its development and/or by prolonging its duration.

Currently, non-methylated cytidyl guanosyl oligodeoxynucleotides (CpG ODNs) are considered to be the most preferred specific group of immunopotentiating compounds capable of inducing an immune response against tumor-specific self-antigens.

These cytidyl guanosyl oligodeoxynucleotides act as toll-like receptor 9 (TLR9) agonists. CpG motifs stand out because of their preferential induction of Th1 responses and tumor-specific CD8⁺ T lymphocytes. TLR9 is predominantly expressed by B cells and dendritic cells (DC) that internalize and directly respond to CpG motifs. Upon triggering of TLR9, DCs mature and migrate to draining lymph nodes where they present antigens to T and B lymphocytes. Importantly, these DCs acquire the unique ability to present captured antigens on MHC class I molecules, a process known as cross-presentation, which is crucial for efficient priming of tumor-specific CTLs. As such, CpG ODN administration has been reported to prevent tumor outgrowth in a prophylactic setting and could also eradicate established tumors in mice. Nierkens, S. et al. (Cancer Res. 68: 5390-5396 (2008)) and by Roux, S. et al. (Cancer Immunol Immunoth. 57: 1291-1300 (2008))

Nevertheless, although the prevention of tumor outgrowth and eradication of established tumors is significant, it is not seen in all animals treated.

Therefore, there is an ongoing search for ways to further increase the level of efficacy of CpG ODNs.

The present invention provides means to increase the level of efficacy of CpG ODNs.

It was now surprisingly found, that when CpG ODNs are combined with 3′,5′-cyclic diguanylic acid (often referred to as cyclic di-GMP or briefly as c-di-GMP), a surprisingly higher immunopotentiating effect of these two components is seen, resulting in a significant improvement of the survival rate. Given the key role of TLR9 and its agonists in the induction of an immunological response to tumor-specific antigens it is indeed surprising that c-di-GMP, that bears no relation to the TLR9-mechanism at all, nevertheless turned out to be very suitable for inducing an immunological response to tumor-specific self-antigens when combined with CpG ODNs.

Cyclic di-GMP is an intracellular signaling molecule, present in multiple bacterial species (Amikam, D et al., J. Bacteriol. 171: 6649-6655 (1989), Ross, P. et al., Nature 325: 279-281 (1987) and D′Argenio, D. A. et al., Microbiology 150: 2497-2504 (2004)).

Cyclic di-GMP is capable of stimulating enhanced protective innate immunity in mammals against various bacterial infections (Ogunniyi, A. D. et al., Vaccine 26: 4676-4685 (2008) and Karaolis D. K. R. et al., Inf. And Immun. 75: 4942-4950 (2007)).

The use of c-di-GMP as an immunopotentiator for a vaccine was i.a. described recently by Gray, P. M. et al., (Cellular Immunology 278:113-119 (2012)). In this publication, a comparison was made between c-di-GMP and the other known vaccine immunopotentiators LPS, CpG ODN and a conventional aluminum salt based immunopotentiator.

However, the use of c-di-GMP is mainly known in the art in the context of classical non-self vaccination: as an immunopotentiator in vaccines comprising bacterial pathogens. A paper has been published that describes the inhibitory effect of c-di-GMP on basal and GF-stimulated human colon cancer cell proliferation in vitro. (Karaolis, D. K. R. et al., BBRC 329: 40-45 (2005)).

However, c-di-GMP has never been suggested as an immunopotentiator for use in self-directed immune responses in combination with CpG ODN's, most likely for the reason mentioned above.

CpG ODNs for use in immune stimulation have been described since 1994 (U.S. Pat. No. 6,429,199). CpG-motifs basically have the structure 5′-X₁-C-pG-X₂-3′. The CpG motif 5′-Pu-Pu-CpG-Pyr-Pyr is known to be amongst the most immunopotentiating (Scheule, R. K., Advanced Drug Delivery Reviews 44: 119-134 (2000)). Basically, their length is from 8-80 bases and they contain at least one non-methylated CpG-motif.

Small differences in efficiency in different animal species are frequently seen. Merely as an example; human TLR9 is optimally triggered by the CpG motif G-T-CpG-T-T, whereas mouse TLR9 is more optimally triggered by G-A-CpG-T-T (Krieg, A. M., Nature Medicine 9: 831-835 (2003).

Optimal CpG motifs for seven veterinary and three laboratory species have been described by Rankin, R., et al., in Antisense and Nucleic Acid Drug Development 11: 333-340 (2001). CpG-motifs that efficiently stimulate canine and feline immune cell proliferation are described by Wernette, C. M., et al., in Veterinary Immunol And Immunopath. 84: 223-236 (2002). Applications for CpG-motifs in poultry have been described i.a. by Ameiss, K. A., et al., in Veterinary Immunol And Immunopath. 110: 257-267 (2006).

Further CpG ODNs are described in WO2012/089800, WO2012/160183 and WO2012/160184. CpG ODNs with different CpG-motives are easily commercially available, and if desired they are easily synthesized. Suitable amounts of CpG ODNs can be found i.a. in the publications mentioned above and in the Examples section.

Thus, a first embodiment of the present invention relates to a pharmaceutical composition comprising an immunostimulating amount of at least two immunopotentiators, wherein a first immunopotentiator is a CpG ODN, a second immunopotentiator is 3′,5′-cyclic diguanylic acid (c-di-GMP) and a pharmaceutically acceptable carrier.

With regard to the determination of suitable amounts of c-di-GMP and CpG ODNs, the following can be said.

For c-di-GMP, a very suitable amount would be in the range between 100 μg per kg of weight and 50 mg per kg of weight. An even more suitable amount would be in the range 500 μg-5 mg/kg. Merely as an example: for use in mice, an amount of 30 μg c-di-GMP/mouse (of 50 g) would be a very suitable amount. For use in humans, a comparably suitable amount would be 45 mg of c-di-GMP per human being.

For CpG ODNs, the suitable amount in micrograms depends i.a. on the length of the CpG ODN. The molecular weight of any CpG ODN roughly relates to 303×n, wherein n is the number of nucleotides in the CpG ODN. Merely as an example: 1 mM of a CpG ODN 20-mer would be about 6 μg.

Of course, the suitable amount of a CpG ODN also depends on the formula of the CpG. Very strong immunopotentiating CpG ODNs can be administered in lower amounts than weaker CpG ODNs.

Merely as an indication: a suitable amount of an average immunopotentiating CpG ODN 20-mer such as CpG 1668 (‘5-TCCATGACGTTCCTGATGCT-3’), well-known in the art, would be in the range between 20 μg per kg of weight and 50 mg per kg of weight. A more suitable amount would be in the range 500 μg-5 mg/kg.

For a comparable CpG ODN 40-mer this would mean between 1000 μg-10 mg/kg.

The c-di-GMP and CpG ODNs are usually administered in a pharmaceutically acceptable carrier. The carrier is preferably a liquid in which c-di-GMP and CpG ODN easily dissolve. Very suitable carriers are water and physiological salt (PBS) solutions. Thus, merely as an example: a pharmaceutical composition according to the invention and suitable for the treatment of a human being could e.g. comprise 45 mg of c-di-GMP and 75 mg of CpG ODN in 100 μl of PBS

The Examples provide further information about suitable amounts of c-di-GMP and CpG ODNs. The literature cited provides further examples of suitable amounts of in various circumstances.

Such a pharmaceutical composition comprising an immunostimulating amount of both c-di-GMP and CpG ODN can be used in many ways for the induction of an immune response against tumor-specific antigens. This can e.g. be a use in combination with in vitro cultured tumor cells as described above or a use in combination with the tumor destruction methods mentioned above.

Thus a second embodiment of the present invention relates to a pharmaceutical composition according to the invention for use in the induction of an immune response against tumor-specific antigens.

It was found that the combined administration of c-di-GMP and CpG ODN in or around a tumor, at or around the moment of tumor destruction induces a very significant immunological response to tumor-specific antigens after tumor destruction in situ. This immune response is long-lasting, and significantly stronger than the immune response induced by each individual immunopotentiator. It is therefore very suitable to eliminate metastasized cells, even if such cells have been latently present in the body.

Moreover, this immune response appeared to be sufficiently strong to prevent the multiplication of the same type of tumor cells even if these are deliberately administered in substantial amounts several weeks after the treatment.

Therefore, a preferred form of this embodiment of the invention relates to a pharmaceutical composition according to the invention for use in in situ tumor-destruction therapy comprising the steps of tumor destruction and administration of said pharmaceutical composition.

It goes without saying that the present invention is equally applicable in the field of human and veterinary medicine.

The wording “an immunostimulating amount of an immunopotentiator” should be interpreted in a broad sense. Such an amount of an immunopotentiator is capable of stimulating the immune system. This stimulation can e.g. (but need not necessarily) be reflected by an increase in cytokine production, such as type 1 interferon (IFN) and interleukin 12 (IL12), as shown in the Example section.

Again, as mentioned above, it goes without saying that the present invention is equally applicable in the field of human and veterinary medicine, although it is advisable (though not mandatory) to match the CpG-motif used to the animal species for which the invention is used. This can easily be done on the basis of the publications summarized above.

In principle, the steps of tumor destruction and administration of the pharmaceutical composition according to the invention can be performed at different moments in time or at the same time. Theoretically, however, one would expect that conditioning a tumor with the pharmaceutical composition several days or better a week or even two or more weeks before applying tumor destruction, with the aim of “priming” the immune system, would be the preferred route.

Surprisingly however, it was found that if the administration of the pharmaceutical composition is done after tumor destruction, within days after tumor destruction, preferably within one day, more preferably within 12 hours, even more preferably within 6 hours, still even more preferably within 2 hours after tumor destruction, the level of immunostimulation is better than when the order of the steps is reversed (Nierkens S, den Brok M H, Sutmuller R P, Grauer O M, Bennink E, Morgan M E, Figdor C G, Ruers T J, Adema G J. Cancer Res. 2008 Jul 1; 68(13): 5390-6.

Also very good results are obtained when the administration of the pharmaceutical composition is done between about two hours before the tumor destruction and the moment of destruction. This is because after destruction the neoplastic mass may be more difficult to approach or enter due to destruction-induced changes in its structure.

Administration of the pharmaceutical composition in the interval between two hours before and two hours after tumor destruction is called peri-operative administration.

Therefore, one preferred form of this embodiment relates to a pharmaceutical composition for use in in situ tumor-destruction therapy comprising the steps of tumor destruction and administration of a pharmaceutical composition according to the invention, characterized in that said steps are in the following order:
a. destruction of the tumor and
b. administration of a pharmaceutical composition according to the invention

More preferred forms of this embodiment relate to the steps in the order as mentioned above wherein the administration of the pharmaceutical composition follows within 24 hours, 12 hours or even 6 hours after tumor destruction, in that order of increasing preference.

Another preferred form of this embodiment relates to a pharmaceutical composition for use in in situ tumor-destruction therapy comprising the steps of tumor destruction and administration of a pharmaceutical composition according to the invention, characterized in that said steps are in the following order:
a. peri-operative administration of a pharmaceutical composition according to the invention and
b. destruction of the tumor

With regard to the site or sites of administration of the pharmaceutical composition, the following considerations should be made:

Preferably, the pharmaceutical composition is administrated directly into the neoplastic mass.

Although slightly less preferred, peri-tumoral administration where the pharmaceutical composition is administered at one or more locations around the neoplastic mass is also possible.

Peri-tumoral administration is administration around the tumor, preferably within a distance of 1 centimeter or less from the surface of the tumor. Most preferably peri-tumoral administration takes place at the surface of the tumor. Administration at one side of the tumor is suitable, but preferably the pharmaceutical composition according to the invention is administered at two or more sides around the tumor.

Another, though less preferred administration is subcutaneous administration in the draining area of the neoplastic mass. Finally, intravenous administration, preferably close to the location of the neoplastic mass is possible.

Therefore, the said administration of the pharmaceutical composition takes place by intravenous administration, subcutaneous administration in the draining area of the neoplastic mass, peri-tumoral administration or intra-tumoral administration, in that order of increasing preference.

Another embodiment of the present invention relates to a pharmaceutical composition according to the invention for use in the treatment of cancer in a mammal suffering from cancer.

A preferred form of this embodiment relates to a pharmaceutical composition for use according to the invention, wherein the mammal has been subjected to tumor destruction.

Still another embodiment of the present invention relates to a pharmaceutical composition according to the invention for use in peri-operative administration in the treatment of cancer in a mammal suffering from cancer wherein the mammal will be or has been subjected to tumor destruction.

another embodiment of the present invention relates to a method of treatment of a mammal suffering from cancer, characterized in that said method of treatment comprises the step of administration of a pharmaceutical composition according to the invention.

Again another embodiment of the present invention relates to a method of treatment of a mammal suffering from cancer, characterized in that said treatment comprises the following steps in the following order:
a. in situ destruction of a tumor and
b. administration of a pharmaceutical composition according to the invention.

Still another embodiment of the present invention relates to a method of treatment of a mammal suffering from cancer, characterized in that said treatment comprises the following steps in the following order:
a. peri-operative administration of a pharmaceutical composition according to the invention and
b. in situ destruction of a tumor.

### EXAMPLES

### Example 1

### Mice and Tumor Cells

C57BL/6n mice (6-8 weeks old) were purchased from Charles River Wiga (Sulzfeld, Germany) and maintained under specific pathogen-free barrier conditions at the Central Animal Laboratory (Nijmegen, The Netherlands). Drinking water and standard laboratory food pellets were provided ad libitum and mice were allowed to settle for at least 1 week before random assignment into specific treatment groups. The experiments were performed according to the guidelines for animal care of the Nijmegen Animal Experiments Committee.

The murine melanoma cell line B16F10 (ATCC) was cultured in complete medium (MEM, 5% fetal bovine serum (Greiner Bio-one), 100 U/ml penicillin G sodium and 100 μg/ml streptomycin (Pen/Strep), MEM sodium pyruvate (1 mM), NaHCO₃, MEM vitamins, MEM non-essential amino acids (all from Gibco), 20 μM β-mercaptoethanol (β-ME)).

### Tumor Model and Cryosurgery

B16F10 melanoma cells were suspended in a mixture of PBS and Matrigel (2:1), and 0.5*10⁶ cells in a total volume of 50 μl were injected s.c. at the right femur. When tumor diameters measured between 6-8 mm (generally at day 9-10) they were randomly assigned to treatment groups. Cryo ablation (Cryo) was performed under isoflurane/O₂/N₂O anesthesia using a liquid nitrogen cryo ablation system (CS76, Frigitronics, Shelton, Conn.) of which the tip is cooled by a continuous flow of circulating liquid nitrogen. During 2 treatment cycles of freezing and thawing the tumor was macroscopically frozen, while leaving surrounding healthy tissue intact. To monitor the induction of long-lasting tumor protection, mice were re-challenged with 15*10³ B16F10 cells 40 days after cryo ablation. Re-challenges were injected in 100 μl PBS s.c. on the right flank. Mice were sacrificed when tumor volume exceeded 1000 mm³ or when tumors brake through the skin barrier.

Injection with Immunopotentiators

CpG 1668 (‘5-TCCATGACGTTCCTGATGCT-3’) with total phosphorothioate-modified backbone was purchased from Sigma Genosys (Haverhill, UK). C-di-GMP was synthesized as described by Spehr V, Warrass R, Höcherl K, Ilg T., in Appl. Biochem. Biotechnol. 2011 Oct;165(3-4):761-75. CpG and/or C-di-GMP were peri-tumorally injected in PBS (p.t., 30 μg divided over 2 injections of 20 μl lining the ablated tumor). All injections were done within 30 min. after ablation.

### Cytokine Measurements

Mouse bone marrow dendritic cells were cultured with granulocyte-macrophage colony-stimulating factor (GM-CSF) and harvested at day 7 of culture. 1.2×10⁵ cells were exposed during overnight incubation to the following immunopotentiators: CpG 1668 1 μg/ml, c-GMP 10 μg/ml, c-di-GMP 10 μg/ml. Next, supernatant was carefully harvested and IL12 or type-I IFN production was determined. For IL12 an ELISA method was used according to the manufacturer's instructions (BD Biosciences). Type-I IFN was determined by a standard bioassay using L929 ISRE reporter cells.

### Statistical Analyses

Kaplan Meier survival curves were analyzed using a log rank test. Cytokine data was analyzed using ANOVA with a post-hoc Bonferroni test.

### Results:

As follows clearly from the graphs of FIG. 1, the combination of tumor destruction and the administration of CpG ODN or c-di-GMP as immunopotentiator leads to an increased survival rate (approx. 50%) after 60 days compared to ablation alone (FIG. 1). However, the combination of tumor destruction with the administration of both CpG and c-di-GMP as immunopotentiator leads to an impressive survival rate of >75% after 60 days, stronger than the survival obtained with both individual immmunopotentiators alone.

The analysis of cytokines produced following incubation of DCs with c-di-GMP and/or CpG revealed that the combination of these immunopotentiators results in synergistic production of type I IFN and IL12.

Type 1 IFN is known to be essential for efficient cross-presentation in dendritic cells and other cells in an anti-tumor setting (Diamond, M. S. et al., Journ. Of Experimental Medicine 208: 1989-2003 (2011)).

Interleukin IL12 is a cytokine typically known to drive the immune system towards Th1 responses, which is in general favorable for anti-tumor immunity.

As can be seen in FIG. 2 no IFN or only modest amounts of IFN were produced when medium, the control c-GMP, or CpG was used. The amount of c-di-GMP used in this experiment (10 μg/ml) only led to a production of <50 U/ml IFN. However, when CpG was combined with c-di-GMP a strong synergistic production of IFN was observed of >200 U/ml.

FIG. 3 demonstrates that DCs do not produce IL12 when medium, the control c-GMP, or c-di-GMP alone was used as immunopotentiator. CpG alone led to a modest level of IL12 production, in a concentration-dependent fashion. However, when CpG was combined with c-di-GMP, a synergistic production of IL12 was found.

### LEGEND TO THE FIGURES

FIG. 1. Anti-tumor memory response following ablation combined with C-di-GMP and CpG. Established B16F10 tumors growing subcutaneously on the right femur were treated with cryo ablation alone, or combined with the indicated immunopotentiators Immunopotentiators (30 mg) were injected in 40 μl PBS in the peri-tumoral area following the ablation. Forty days later, naïve and tumor-free mice received a re-challenge with 15.000 B16F10 cells s.c. at the flank. Growth of this re-challenge is depicted as a Kaplan-Meier survival curve demonstrating increased protection from tumor outgrowth after ablation combined with CpG or c-di-GMP, and superior protection when CpG is combined with c-di-GMP. p<0.05 for cryo/c-di-GMP compared to cryo/CpG/c-di-GMP.

FIG. 2. Synergistic type I IFN production by DCs upon combined treatment with c-di-GMP and CpG. Mouse bone marrow dendritic cells were cultured with GM-CSF and harvested at day 7 of culture. 1.2×10⁵ cells were exposed during overnight incubation to the indicated immmunopotentiators or combinations of immunopotentiators: CpG 1 μg/ml, c-GMP 10 μg/ml, c-di-GMP 10 μg/ml. Next, supernatant was harvested and type I IFN production was determined by a standard bioassay using L929 ISRE cells. Results are shown as means with sem, *=p<0.001 vs. all other bars. Similar results were obtained in three independent experiments.

FIG. 3. Synergistic IL12 production by DCs upon combined treatment with c-di-GMP and CpG. Mouse bone marrow dendritic cells were cultured with GM-CSF and harvested at day 7 of culture. 1.2×10⁵ cells were exposed during overnight incubation to the indicated immunopotentiators: CpG 1 μg/ml, c-GMP 10 μg/ml, c-di-GMP 10 μg/ml. Next, supernatant was harvested and IL12 production was determined by standard ELISA methods. Results are shown as means with sem, *=p<0.001. Similar results were obtained in three independent experiments.

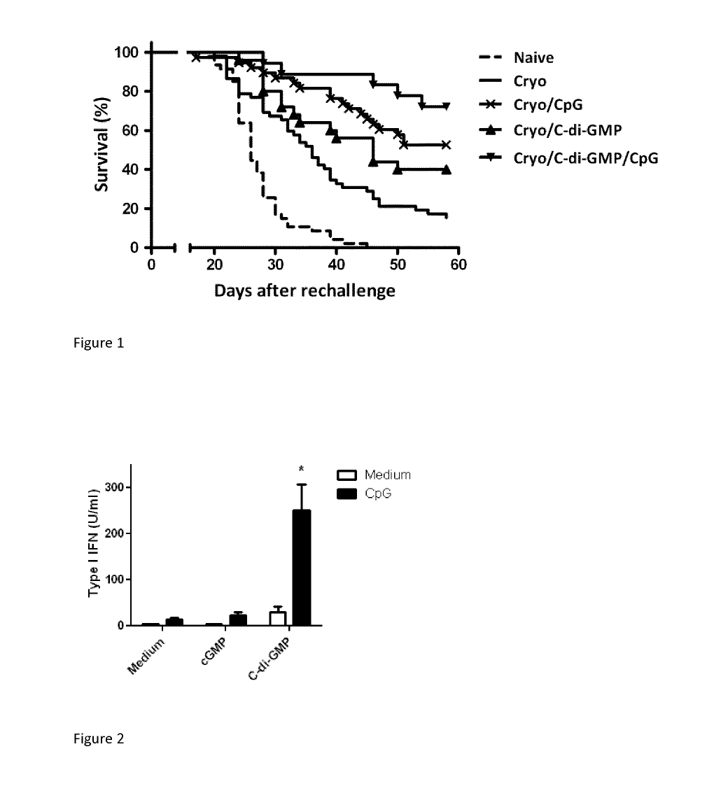

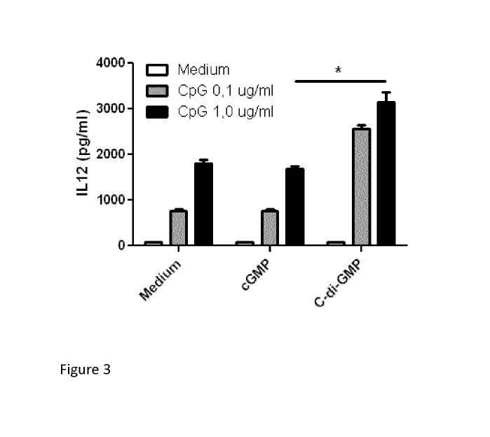

1-15. (canceled)

16. A pharmaceutical composition comprising an immunostimulating amount of at least two immunopotentiators and a pharmaceutically acceptable carrier;
wherein a first immunopotentiator is a non-methylated cytidyl guanosyl oligodeoxynucleotide (CpG ODN); and
wherein a second immunopotentiator is 3′,5′-cyclic diguanylic acid (c-di-GMP).

17. A method of inducing an immune response against tumor-specific antigens in a mammal comprising administering the pharmaceutical composition of claim 16 to the mammal.

18. A method of performing in situ tumor-destruction therapy in a mammal comprising performing tumor destruction on a tumor in the mammal and administering the pharmaceutical composition of claim 16 to the mammal.

19. The method of claim 18 wherein the step of performing tumor destruction on the tumor is performed prior to the step of administering the pharmaceutical composition.

20. The method of claim 19 wherein the step of administrating the pharmaceutical composition follows within 24 hours after the step of tumor destruction.

21. The method of claim 20 wherein the step of administrating the pharmaceutical composition follows within 12 hours after the step of tumor destruction.

22. The method of claim 20 wherein the step of administrating the pharmaceutical composition follows within 6 hours after the step of tumor destruction.

23. A method of performing in situ tumor-destruction therapy in a mammal comprising performing tumor destruction on a tumor in the mammal and administering the pharmaceutical composition of claim 16 peri-operatively to the mammal.

24. The method of claim 17, wherein the mode of administering the pharmaceutical composition is intravenous, subcutaneous in the draining area of the neoplastic mass, peri-tumoral or intra-tumoral.

25. A method of treating a mammal suffering from cancer comprising administering the pharmaceutical composition of claim 16 to the mammal.

26. A method of treating a mammal suffering from cancer, comprising performing tumor destruction on a tumor in the mammal and administering the pharmaceutical composition of claim 16 peri-operatively to the mammal.

27. The method of claim 26 wherein the step of performing tumor destruction on the tumor is performed prior to the step of administering the pharmaceutical composition.

28. The method of claim 26 wherein the step of performing tumor destruction on the tumor is performed after the step of administering the pharmaceutical composition.


SEQUENCE LISTING (1 sequence)

SEQ ID NO: 1 - DNA, 20 bp, artificial sequence
  Artificial immunopotentiator
tccatgacgttcctgatgct